(19) United States
(12) Patent Application Publication
(10) Pub. No.: US 2007/0060962 A1
(43) Pub. Date: Mar. 15, 2007

(54) Apparatus and methods for cardiac resynchronization therapy and cardiac contractility modulation

(76) Inventor: Carlo Pappone, Milano (IT)

Correspondence Address: Edward Renner, Suite 400, 7700 Bonhomme, St. Louis, MO, 63105, US

(21) Appl. No.: 11/478,442

(22) Filed: Jun. 29, 2006

Related U.S. Application Data
(60) Provisional application No. 60/702,487, filed on Jul. 26, 2005.

(51) Int. Cl. A61N 1/362

(52) U.S. Cl. 607/9

(57) ABSTRACT

A method of placing and testing placement of a plurality of pacing and stimulation leads for cardiac resynchronization therapy (CRT) employs cardiac contractility modulation (CCM), using a plurality of cardiac contractility modulation leads placed at a plurality of sites.

### CROSS-REFERENCE TO RELATED APPLICATIONS

This application claims the benefit of U.S. Provisional Patent Application Ser. No. 60/702,487, filed Jul. 26, 2005, the entire disclosure of which is incorporated herein by reference.

### BACKGROUND OF THE INVENTION

Cardiac contractility modulation (CCM) stimulation has been used in pacing the hearts of cardiac patients. In the performance of CCM, low-impedance bi-polar electrical leads typically are placed in the heart and activated to improve heart function. High current through low-impedance leads, however, tends to drain the battery life of implanted devices. Additionally, when bi-polar leads are used, proximal electrodes can bleed current into the blood pool. Cardiac response to CCM is dose-dependent. Although the application of eighteen mA of current in one location could produce an optimal response, it could also exceed a sensory threshold and cause pain in the patient.

### SUMMARY OF THE INVENTION

The present invention, in one implementation, is directed to a method of stimulating sites in the heart of a patient. One or more cardiac resynchronization therapy (CRT) leads are placed in one or more cardiac sites. A plurality of cardiac contractility modulation (CCM) leads are placed in a plurality of cardiac sites. The sites are selectively stimulated.

In another implementation, an apparatus for stimulating sites in the heart of a patient includes a plurality of cardiac resynchronization therapy (CRT) and cardiac contractility modulation (CCM) leads positioned in a plurality of cardiac sites. A pulse generator device is configured to stimulate one or more of the sites via the leads during a local refractory period.

Some embodiments of the present invention provide for placement and/or testing placement of leads for cardiac resynchronization therapy (CRT) using cardiac contractility modulation (CCM). A plurality of CCM leads are placed in a plurality of cardiac sites.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a schematic diagram showing exemplary placement of a CRT lead for pacing and sensing in accordance with one implementation of the invention;

FIG. 2 is a flow diagram of a method of stimulating cardiac sites of a patient in accordance with one implementation of the invention; and

FIG. 3 is a block diagram of an apparatus for stimulating cardiac sites of a patient in accordance with one implementation of the invention.

### DETAILED DESCRIPTION OF THE PREFERRED EMBODIMENTS

The following description of the preferred embodiment(s) is merely exemplary in nature and is in no way intended to limit the invention, its application, or uses.

In some implementations of the invention, cardiac sites may be stimulated by placing one or more cardiac resynchronization therapy (CRT) leads in one or more cardiac sites, placing a plurality of cardiac contractility modulation (CCM) leads in a plurality of cardiac sites, and selectively stimulating the sites. In some implementations of the invention, a pulse is delivered to the heart at a level above a sensory threshold during the local refractory period to produce an effect of “priming” the cellular ion channels for quicker re-polarization. Such stimulation is in contrast to commonly used stimulations which are delivered during the post-refractory period.

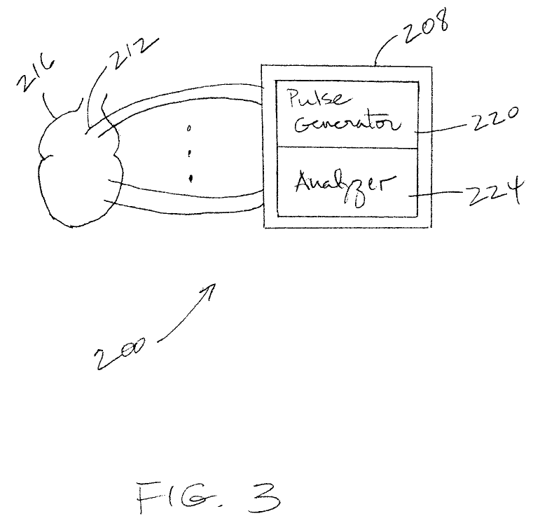

An exemplary configuration of an apparatus for stimulating cardiac sites of a patient is indicated generally in FIG. 3 by reference number 200. An external pulse generator device 208 is connected with a plurality of cardiac stimulation leads 212 positioned in a patient's heart 216. The device 208 includes a programmable pulse generator 220 and an analyzer 224. The device 208 may be used for evaluating cardiac lead placement sites as further described below. The device 208 can be programmed to stimulate a plurality of cardiac sites simultaneously or sequentially. The device 208 may be programmed with a delay for sequential stimulation during the local refractory period.

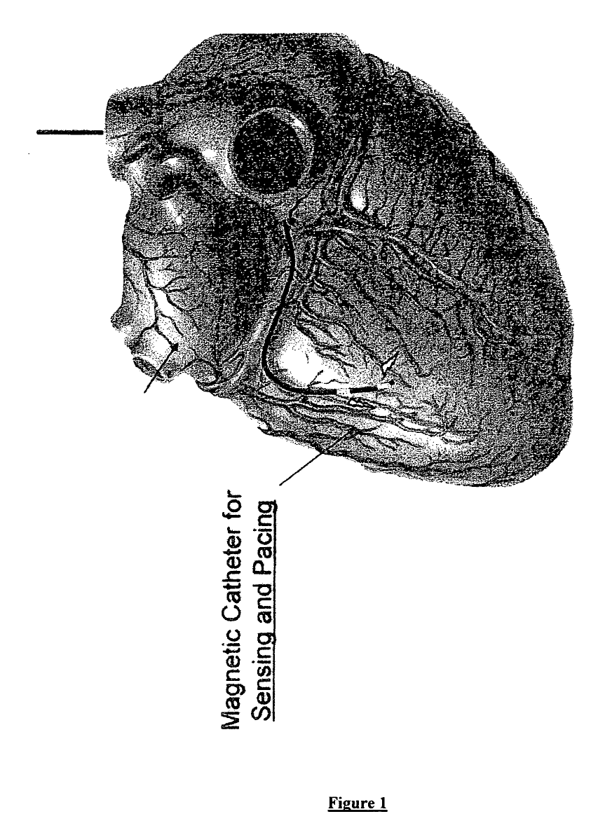

In one exemplary method for testing cardiac resynchronization therapy (CRT) and CCM sites, a plurality of CCM leads may be placed as follows. A bipolar pace/sense lead for CCM stimulation is placed in the right atrium (RA). A bipolar pace/sense lead is placed in the right ventricle (RV). A bipolar CCM stimulation lead is placed on the high-mid right anterior ventricular septum or in the anterior medial area of the left coronary venous system. A bipolar CCM stimulation lead is placed in the posterior area of the left coronary venous system, and a bipolar CCM stimulation lead is placed in the lateral area of the left coronary venous system. One or more CRT leads may be placed as is usually done in the coronary sinus venous vasculature. FIG. 1 shows an example of a CRT lead placed in such a manner for pacing and sensing, where the pacing lead itself takes the form of a magnetically enabled device.

A high right septal CCM lead may be a screw-in lead having a fixed or retractable screw. Leads may include magnets and/or be placed over guidewires for direction by magnetic field. The CCM leads are thin relative to CRT leads and of high impedance as further described below. CRT leads may be placed using magnets with or without guidewires. Screw-in electrodes may be screwed into position using magnetic fields. A CRT lead may be stimulated in a typical manner. The CCM leads may be stimulated, for example, using a bi-phasic square wave having a pulse duration between 0 and 60 ms, e.g., 20 ms. The wave may have an amplitude between 5.0 and 10.0 volts, delivered between 20 and 50 ms post-local activation sense, to stimulate during the refractory period, with about 15 mA delivered to the high right septal lead and about 6 mA in each of the left CCM leads. “High impedance”, when used in reference to CCM leads, means enough impedance to result in the foregoing currents through the leads. The leads alternatively could be placed using remote mechanical guidance systems in place of remote magnetic guidance systems.

The device 208 can be used in performing implementations of the foregoing method. The device 208 may be used to stimulate a plurality of sites simultaneously or sequentially with a programmable delay for sequential stimulation during the local refractory period. When used for evaluating cardiac contractility modulation (CCM) lead placement sites, the device 208 may be configured to stimulate the sites with one or more output ranges of between 3- and 20-mA energy delivery (bi-phasic square wave, 0-60 ms pulse duration with 20 ms exemplary, 5.0-10.0 V, delivered 20-50 ms post-local activation sense). The device 208 can be configured to stimulate a plurality of sites simultaneously or sequentially, with a programmable delay for sequential stimulation for evaluating multiple CRT lead placement sites in a voltage output range and pulse width range common to implantable devices. The device 208 can be programmed to perform sensing and pacing of right atrium, right ventricle (RV) and left ventricle (LV) sites. For example, the device 208 can be triggered from atrium-sense or atrium-pace, right-ventricle-pace or right-ventricle-sense, in programmable fashion. The device 208 can be used to stimulate and evaluate CRT and/or CCM sites in the same evaluation or can be used to stimulate and evaluate CRT or CCM sites independently. The device 208 may be used with a magnetic navigation system or a mechanical navigation system, and with or without a pressure-volume (PV) loop recorder.

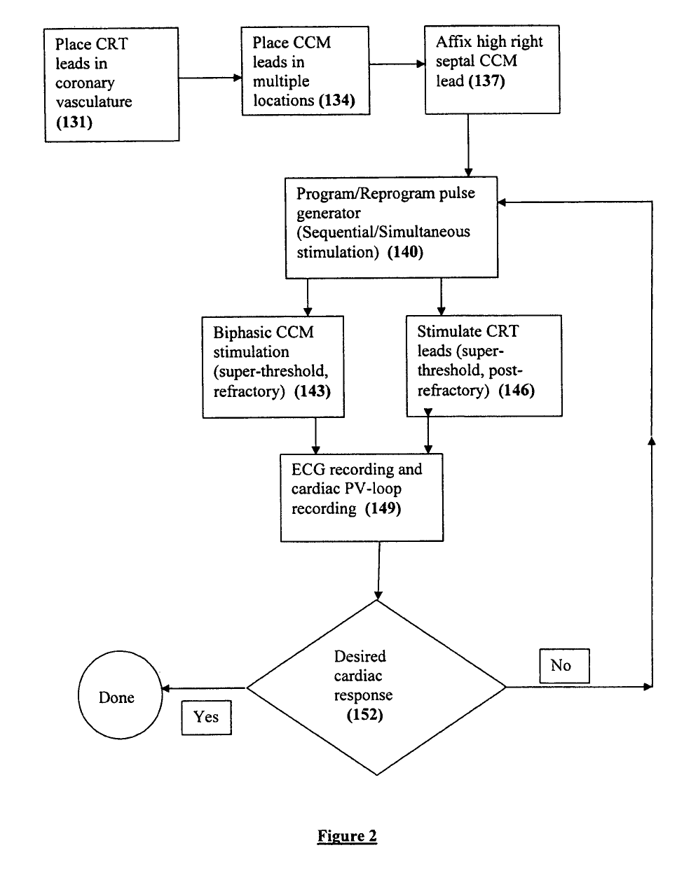

One implementation of a method of stimulating cardiac sites is indicated generally in FIG. 2 by reference number 100. The device 200 may be used to implement the method 100 to perform cardiac pacing. At least one bipolar CRT lead is navigated to and placed in the coronary sinus vasculature and affixed by means of a suitable fixation device in step 131. A plurality of bipolar leads are placed for cardiac contractility modulation (CCM) at several cardiac locations in step 134. For example, these locations could be one or more of a right atrial location, a right ventricle location, a high-mid right anterior ventricular septal location (e.g., as described in step 137), an anterior medial area of the left coronary venous system, a posterior area of the left coronary venous system, and a lateral area of the left coronary venous system. In the present example, a high right septal lead is affixed at its location in step 137.

The device 208 is suitably programmed in step 140 by the user to generate a series of sequential or simultaneous stimulation pulses. Specifically and for example, the CCM leads are stimulated using bi-phasic square wave pulses of pulse duration in the range 0-60 ms, voltage amplitude 5-10 V and delivered 20-50 ms post-local activation, i.e., in the refractory period. The left CCM leads carry, e.g., about 6 mA of current, and the high mid-septal CCM lead carries, e.g., a current of about 15 mA. At least one of these leads could be delivered by a remote navigation system that is magnetically or mechanically actuated.

CCM stimulation pulses are suitably applied in step 143 and CRT stimulation is suitably applied in step 146. In step 149 the ensuing cardiac ECG activity, and more specifically intra-cardiac ECG activity, is recorded and observed. Additionally or alternatively, the cardiac pressure-volume loop (PV loop) is recorded with a standard PV-recording catheter device. In step 152 the cardiac response is studied and if the improvement in cardiac activity is deemed satisfactory, the procedure is finished; otherwise the pulse generator 220 may be reprogrammed in step 140 to deliver a different sequence of stimulation pulses, possibly with at least one of the leads being moved to an alternate location.

The description of the invention is merely exemplary in nature and, thus, variations that do not depart from the gist of the invention are intended to be within the scope of the invention. Such variations are not to be regarded as a departure from the spirit and scope of the invention.

1. A method of placing and testing placement of a plurality of pacing and stimulation leads for cardiac resynchronization therapy (CRT) employing cardiac contractility modulation (CCM), where a plurality of cardiac contractility modulation (CCM) leads are placed at a plurality of sites.

2. The method of claim 1 where a bipolar CCM stimulation lead is placed in the right atrium.

3. The method of claim 1 where a bipolar pacing/sensing lead is placed in the right ventricle.

4. The method of claim 1 where a bipolar CCM stimulation lead is placed on the high-mid right anterior ventricular septum.

5.-7. (canceled)

8. The method of claim 4 where the high-mid right anterior ventricular septal bipolar CCM stimulation lead is a screw-in lead.

9.-12. (canceled)

13. The method of claim 1, where at least one of the CCM leads is stimulated using a bi-phasic square wave pulse with a pulse duration of less than about 65 ms.

14. The method of claim 1, where at least one of the CCM leads is stimulated using a bi-phasic square wave pulse with a voltage amplitude of less than about 10 V.

15. (canceled)

16. The method of claim 1, where the stimulation with at least one of the CCM leads is delivered in the post-local activation refractory period at its placement site about 20-50 ms post-local activation.

17.-19. (canceled)

20. A programmable pulse generator device with analyzer that can stimulate a plurality of sites sequentially with a programmable delay for sequential stimulation during the local refractory period for evaluating a plurality of cardiac contractility modulation lead placement sites.

21. (canceled)

22. The pulse generator of claim 20, where the pulse output delivers between about 3 mA and about 20 mA of current.

23. The pulse generator of claim 20, where the pulse duration is less than about 60 ms.

24. The pulse generator of claim 20, where the pulse voltage amplitude is less than about 10 V.

25. The pulse generator of claim 20, where the pulse is delivered in the post-local activation refractory period.

26. The pulse generator of claim 20, where the pulse is delivered in the post-local activation refractory period about 20-50 ms post-local activation.

27.-55. (canceled)

56. A method of stimulating sites in the heart of a patient, the method comprising:
placing CCM stimulation leads in the right atrium and right ventricle;
placing a CCM stimulation lead in the high-mid right anterior ventricular septum or in the anterior medial area of the left coronary venous system;
placing CCM stimulation leads in the posterior area of the left coronary venous system and lateral area of the left coronary venous system;
placing one or more CRT leads in the coronary sinus venous vasculature; and
selectively applying signals to the heart through the leads.

57. The method of claim 56, wherein one or more of the CCM leads are bipolar.

58. The method of claim 56, wherein placing a CCM stimulation lead in the high-mid right anterior ventricular septum or the anterior medial area of the left coronary venous system comprises screwing in the lead.

59. The programmable pulse generator of claim 20 further comprising one or more cardiac resynchronization therapy (CRT) leads and a plurality of bipolar cardiac contractility modulation (CCM) leads that are adapted to be positioned in a plurality of cardiac sites, wherein the pulse generator device is configured to stimulate one or more of the sites via the leads during a local refractory period.

60. The apparatus of claim 59, wherein the pulse generator device is configured to pace right atrium, right ventricle and left ventricle sites.

61. The apparatus of claim 59, wherein the pulse generator device is configured to evaluate CRT and CCM sites in the same evaluation.

62. (canceled)
